(19) United States
(12) Patent Application Publication
(10) Pub. No.: US 2026/0317725 A1
(43) Pub. Date: Oct. 15, 2026

(54) SYSTEMS AND METHODS FOR TISSUE PROCESSING

(71) Applicants: INSTITUT NATIONAL DE LA SANTÉ ET DE LA RECHERCHE MÉDICALE, Paris (FR); UNIVERSITE DE LILLE, Paris (FR); INSTITUT PASTEUR DE LILLE, Lille (FR); CENTRE HOSPITALIER UNIVERSITAIRE DE LILLE, Lille (FR)

(72) Inventors: François PATTOU, Lille Cedex (FR); Julie KERR-CONTE, Lille Cedex (FR); Naziha MENASRIA, Lille Cedex (FR)

(21) Appl. No.: 19/484,143

(22) PCT Filed: May 21, 2024
(86) PCT No.: PCT/EP2024/063897

§ 371 Date: Nov. 13, 2025

Related U.S. Application Data
(60) Provisional application No. 63/503,204, filed on May 19, 2023.

(51) Int. Cl. C12N 5/071; A61K 35/39

(52) U.S. Cl. CPC C12N 5/0676; A61K 35/39; C12N 2509/10

(57) ABSTRACT

Provided are closed systems methods of use for isolation of mammalian tissues.

### TECHNICAL FIELD

The instant disclosure relates to methods for tissue isolation and processing. This disclosure pertains in particular to the field of pancreatic cell isolation and processing, for example for the treatment of diabetes.

### BACKGROUND

It has been proposed to isolate and transplant human pancreatic islet cells as a therapy for diabetes. Human islet isolation based on the Ricordi procedure has not changed since 1990. The procedure is based on the enzymatic digestion of pancreatic tissue, washing to remove enzymes, and purification of the 1-2% of low-density islets from contaminating dense exocrine tissue using density gradients. After extensive washing, islets are cultured in flasks, and manual changes of culture medium are performed every 12-24 h, before manually collecting islets for conditioning in a blood bag for transplantation.

While effective, the procedure involves the use of equipment that is being discontinued. For example, The COBE 2991 routinely used in traditional blood banks for hematopoietic stem cell processing was repurposed for islet purification with a mean islet recovery post purification of >60%. Some refrigerated COBE 2991 prototypes were made (<20), whereas most islet isolation centers turned to alternative methods to refrigerate the COBE 2991, including working in a cold room.

In August 2020, Terumo Blood and Cell Technologies announced that due to changes in “EU Medical Device Regulation” regulatory legislation (EU MDR 2017/7045) the COBE cell processor and disposables would no longer be available in Europe (including the UK), countries in the Middle East and Africa (MEA). There is no current alternative solution; thus, an alternative must be identified so that islet isolation centers in Europe and the Middle East may continue operation. Effective Dec. 1, 2020 service will be discontinued on COBE 2991 with serial numbers 2000 and below (ref letter TERUMO December 2018); service contracts will no longer be renewed as of Dec. 1, 2019. End of support for spares, repairs, service agreements, and technical services (final sunset date) will be Dec. 29, 2025. A similar letter from Terumo's global product manager—Cell Therapy Technologies—in April and October 2023 announced the end of service of the COBE cell processor, and discontinuation of consumables and support in the world by Mar. 31, 2031 (final sunset date).

Further, while the COBE system has been described as a “closed” system (i.e. a system wherein processing is isolated from the surrounding environment), it is not a true closed system. In particular, in the islet cell purification procedure, the gradients in most centers are used in an open phase gradient maker, and islets are systematically collected in centers in an open phase and not directly in bags as required by a closed system and as proposed herein.

In addition, the COBE 2991 only performs one step of the islet manufacturing: the purification on density gradients. A centrifuge (or 2) is still required in the cleanroom for the other steps (washing, volume reduction, resuspension in another medium). An adequate staff is required when performing this procedure (a team of 3-4 trained staff for >6 hours), and the reconfigurated COBE with a refrigeration unit generates a turbulence and particles (due to the heat) that impact the cleanroom Good Manufacturing Practice (GMP) environment.

Thus, improvement is needed in this area. COBE, around for 50 years and many COBE 2991 machines in service for >30 years, the end creates an opportunity to identify more adapted technology that can be exploited not only for the density purification step but also during other key steps of the process.

While the use of the SEPAX™ is known, the traditional technique used in the SEPAX™ with use of one standard density gradient as is performed for other applications, in particular hematopoetic cells, will not optimally work to purify heterogenous pancreatic tissue.

### SUMMARY

The instant disclosure provides systems, devices, methods, a workflow, and kits for use in tissue isolation and processing.

In one aspect, the instant disclosure provides a method of isolating pancreatic cells, comprising: obtaining a suspension comprising biological material including pancreatic cells; identifying an optimum medium for purifying the pancreatic cells; separating the pancreatic cells from the rest of the biological material using the identified optimum medium, thereby isolating the pancreatic cells; wherein identifying the optimum medium for purifying the pancreatic cells comprises performing a density gradient centrifugation on a portion of the suspension using a plurality of media, and identifying the optimum medium as the medium comprising pancreatic cells or the medium below an interface comprising pancreatic cells, wherein separating the pancreatic cells comprises performing a purification using the optimum medium, thereby purifying the pancreatic cells.

In some embodiments, the pancreatic cells may be islet cells.

In some embodiments, the biological material may be obtained through extraction from at least part of a pancreas or through differentiation of stem cells.

In some embodiments, the biological material may be obtained through extraction, and wherein obtaining a suspension comprises: digesting at least part of the pancreas to obtain a digestate; and reducing the digestate in volume, washing the reduced digestate in a wash medium, and resuspending the reduced digestate in the wash medium or another medium such as University of Wisconsin, UW, solution In some embodiments, reducing the digestate in volume and washing the reduced digestate may be performed in a SEFIA Select™ machine.

In some embodiments, obtaining a suspension may further comprise dividing the resuspended digestate in one or a plurality of bags for subsequent use, storage or preincubation.

In some embodiments, density gradient centrifugation on a portion of the suspension may be performed using a discontinuous gradient

In some embodiments, the discontinuous gradient of the portion of the suspension may comprise a plurality of fractions of different densities In some embodiments, seven fractions may be used.

In some embodiments, density gradient centrifugation on a portion of the suspension may use a dye, and the dye may be dithizone.

In some embodiments, density gradient centrifugation on a portion of the suspension may be performed using top loading or bottom loading of the portion of the suspension in the plurality of media.

In some embodiments, identifying the optimum medium for purifying the pancreatic cells may comprise identifying a second medium distinct from the optimum medium, the second medium comprising pancreatic cells or being a second medium below a second interface comprising pancreatic cells

In some embodiments, the purification may be performed in a machine functioning using a rotating syringe principle, such as a SEPAX C-Pro Cell Processing machine or a Sefia S2000 machine of Cytiva Europe.

In some embodiments, the method may further comprise cooling the pancreatic cells.

In some embodiments, the method may further comprise conditioning for transplantation or usage the purified pancreatic cells in a form suitable for transplantation, on site or at a distant center.

In another aspect, the present disclosure provides a system for applying the above method.

In another aspect, the present disclosure provides a method of treating a patient in need thereof, comprising administering a therapeutically effective amount of pancreatic cells isolated by the method described above.

Another aspect of the present disclosure is a system of carrying out the method described above. Such system may be a “closed” system, for example a Good manufacturing Practice Compliant (cGMP-compliant) system, for example a SEPAX™/SEFIA™ system. In embodiments, multiple process steps are performed using the same system or device, for example the SEPAX™/SEFIA™ systems.

### DRAWINGS

The invention will be described in further details with reference to the following illustrative drawings, among which:

FIG. 1 is a diagram showing the steps of the method of isolating pancreatic cells according to the invention;

FIG. 2 is a diagram showing the steps of obtaining a suspension of the method of FIG. 1; and

FIG. 3 is a diagram showing the steps of identifying an optimum medium of the method of FIG. 1 on a portion of the suspension.

FIG. 4 is a diagram showing the steps of conditioning the purified pancreatic cells of FIG. 1, for example for transplantation.

### DETAILED DESCRIPTION

The instant disclosure provides a multi-step protocol comprising systems, for example, “closed” systems, for example cGMP-compliant systems, to conduct islet cell processing. For example, in disclosed embodiments, the system comprises a SEPAX™ system. Currently, the SEPAX™ platform is used for the processing of hematopoietic stem cells from mobilized peripheral. Disclosed embodiments comprise use of the SEPAX™ platform to isolate and process mammalian islet cells, for example human islet cells.

### Definitions

“A” and “an” are used herein to refer to one or to more than one (i.e., to at least one) of the grammatical object of the article. By way of example, “an element” means one element or more than one element.

“Comprise,” “comprising,” “include,” “including,” “have,” and “having” are used in the inclusive, open sense, meaning that additional elements may be included. The terms “such as”, “e.g.”, as used herein are non-limiting and are for illustrative purposes only. “Including” and “including but not limited to” are used interchangeably.

“Effective,” “effective amount,” and “therapeutically effective amount” refer to that amount of a pharmaceutical composition thereof that produces a beneficial result after administration.

“In vitro” refers to an artificial environment and to processes or reactions that occur within an artificial environment. In vitro environments include, but are not limited to, test tubes and cell culture. The term “in vivo” refers to the natural environment (e.g., an animal or a cell) and to processes or reaction that occur within a natural environment.

“Or” as used herein should be understood to mean “and/or”, unless the context clearly indicates otherwise.

“Patient,” “subject,” or “host” to be treated by the subject method can mean either a human or non-human animal, such as a mammal, a fish, a bird, a reptile, or an amphibian.

“Pharmaceutically acceptable” or “therapeutically acceptable” refers to a substance which does not interfere with the effectiveness or the biological activity of the active ingredients and which is not toxic to a patient

“Treatment” or “treating” refers to any therapeutic intervention in a mammal, for example a human or animal such as a companion animal, including: (i) prevention, that is, causing the clinical symptoms not to develop, e.g., preventing infection or inflammation from occurring and/or developing to a harmful state; (ii) inhibition, that is, arresting the development of clinical symptoms, e.g., stopping an ongoing infection so that the infection is eliminated completely or to the degree that it is no longer harmful; and/or (iii) relief, that is, causing the regression of clinical symptoms, e.g., causing a relief of fever and/or inflammation caused by or associated with a microbial infection. Treatment can comprise multiple administrations of compositions disclosed herein.

“Reducing”, “suppressing” and “inhibiting” have their commonly understood meaning of lessening or decreasing.

### Method and System

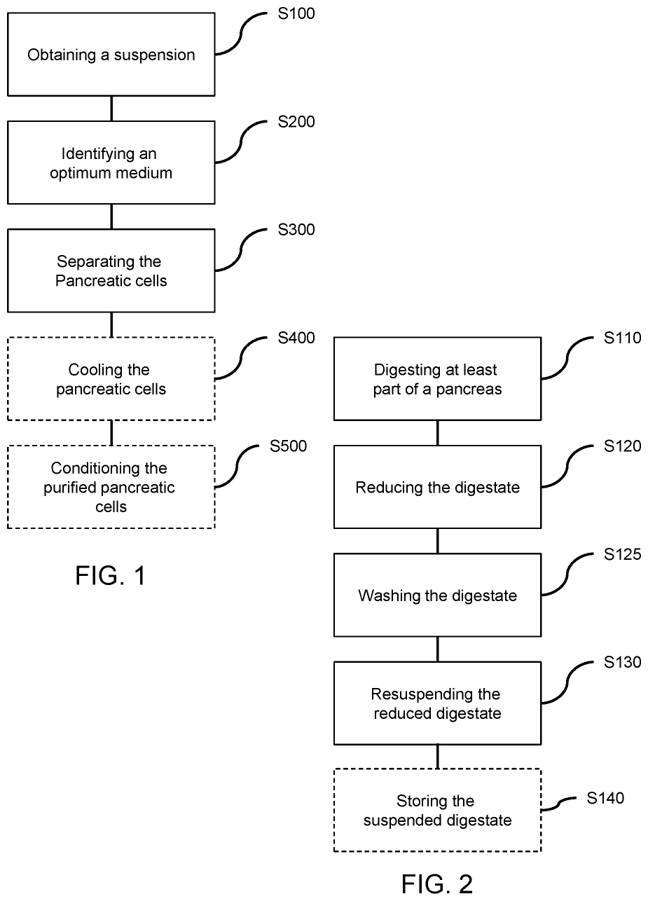

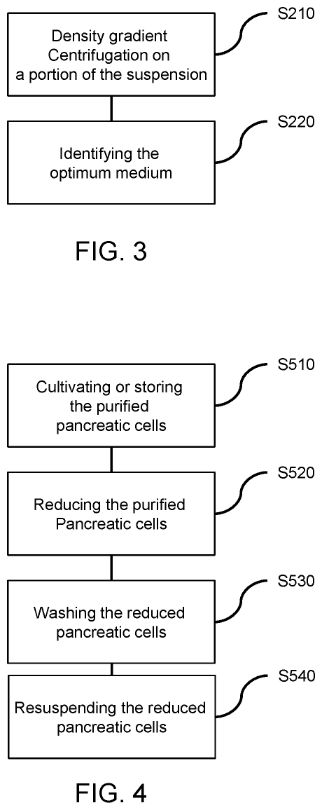

As shown in FIG. 1, the instant disclosure provides a method of isolating pancreatic cells. The method may be carried out in a GMP cleanroom environment as per ISO 14644-1 standard.

The method comprises:

obtaining S100 a suspension comprising biological material including pancreatic cells;
identifying S200 an optimum medium for purifying the pancreatic cells; and
separating S300 the pancreatic cells from the rest of the biological material using the identified optimum medium, thereby isolating the pancreatic cells. In some examples, the pancreatic cells are islet cells.

The biological material may be obtained through differentiation of stem cells. Alternatively, the biological material may be obtained through extraction of at least part of the pancreas. In such a case, obtaining S100 a suspension may comprise digesting S110 at least part of a pancreas to obtain a digestate, reducing S120 the digestate in volume, and washing and resuspending S130 the reduced digestate.

Digesting S110 the at least part of a pancreas may be performed using enzymes, for example crude collagenase or purified or recombinant collagenase and neutral protease. The method used may be the one described by Ricordi et al. [C1]. In some cases, a whole pancreas is digested. In other cases, only a portion thereof is digested.

Reducing S120 the digestate in volume may comprise collecting mixed tissue and cells in bags (2-10 L) and reducing the volume from for example from between 8 and 10 litres to 50 to 220 mL. In some cases, reducing S120 the digestate may be performed in an automated closed system, such as a Sefia Select™ machine from Cytiva Europe. By closed system, it is to be understood a system wherein the digestate does not come into contact with the external environment during the reduction of the digestate. The closed system minimizes the risk of contamination and maintain cell viability.

Washing S125 the reduced digestate may comprise introducing a wash medium adapted for removing enzymes from the digestate by dilution. For example, the wash medium may be simple salt solutions for example Hank's salt solution, or culture medium such as M199, MEM, CMRL 1066, or any medium compatible with pancreatic cells. In some cases, washing S125 the digestate in the wash medium may also be performed in an automated closed system, such as a Sefia Select™ machine from Cytiva Europe.

Resuspending S130 the reduced digestate may be performed in the wash medium, in University of Wisconsin (UW) solution, or other solutions which improve density gradient separation prior to purification. Resuspending S125 the digestate may also be performed in an automated closed system, such as a machine from Cytiva Europe.

Obtaining S100 a suspension may further comprise storing S140 the suspended digestate in one or a plurality of bags, such as blood bags. Storing the suspended digestate may be done before purification.

Identifying S200 an optimum medium for purifying the pancreatic cells comprises performing S210 a density gradient centrifugation on a portion of the suspension using a plurality of media and identifying S220 the optimum medium as the medium comprising pancreatic cells or the medium below an interface comprising pancreatic cells.

By medium, it is to be understood a solution with a given density configured to form a density gradient. A plurality of media of different densities may be combined to form the density gradient. By a portion of the suspension, it is to be understood that a small part of the total volume of the suspension is isolated from the suspension for performing S210 a density gradient centrifugation.

Identifying S200 the optimum medium makes it possible to adapt the method for each cell donor and, in particular, the step of separating S300 the pancreatic cells from the rest of the suspension. The optimum medium maximizes recovery of the desired pancreatic cells during separating S300 the pancreatic cells from the rest of the suspension, and enhances purification. The pancreatic cells may be recovered in a volume compatible with their subsequent use, such as transplantation(<10-15 ml). Further, identifying S200 the optimum medium prepares for multiple purification procedures of the suspension, for example if the suspension volume is important (>30 ml). Large volume suspensions (>30 ml) will decrease pancreatic cell recovery after purification and thus require simultaneous or sequential purification processes.

The S210 density gradient centrifugation may be performed on the portion of the suspension using a continuous or discontinuous gradient. Preferably, a discontinuous gradient is used. For example, the discontinuous gradient may be obtained using a plurality of media, each media having a different density. For example, the plurality of media may be Ficoll based or iodixinol based fractions, for example 7 fractions, of different densities spanning between 1.000 to 1.120 or even 1.132. A final fraction of Hanks' Balanced Salt Solution (HBSS), culture medium or UW may be loaded on top of the fractions. The dye used for the density gradient centrifugation may be dithizone. It may be added to a fraction of the suspension in a 15 ml tube to rapidly color a portion of the pancreatic cells, in particular within 2-5 minutes. The portion of the suspension can either be top loaded directly on top of the gradient, or the portion of the suspension can be bottom loaded. Following S210 density gradient centrifugation (program 600 g×4-6 minutes) in a cooled centrifuge, pancreatic cells are coloured and visible, and separated from the predominantly exocrine fractions.

Identifying S220 is performed after the centrifugation. Identifying S220 may be performed by visually detecting coloured layers. The coloured layers indicate the presence of the pancreatic cells. The coloured layers are formed in a medium of a particular density or at an interface between two media of different densities, depending on the density of the pancreatic cells. In some cases, only one medium or interface is identified as containing pancreatic cells. In such case, the medium comprising the coloured layer or the medium below the interface is identified as the optimum medium. In other cases, more than one medium or interface is identified as containing pancreatic cells. In this latter case, the medium of the lowest density comprising pancreatic cells or the medium below the lowest (i.e. most dense) interface is identified as the optimum medium.

Alternatively, when more than one layer or interface is identified as containing pancreatic cells, identifying S220 may comprise identifying a second medium distinct from the optimum medium, the second medium also comprising pancreatic cells or being a second medium below a second interface comprising pancreatic cells. In this case, two medias are selected for subsequent purification S300, further enhancing pancreatic cell recovery.

Separating S300 the pancreatic cells comprises performing a purification using the optimum medium identified during identifying S200. Separating S300 thereby purifies the pancreatic cells, which can be recovered. In the case where only the optimum medium is used, the pancreatic cells may be recovered in one layer. If the optimum medium and the second medium is used, two separate layers comprising pancreatic cells may be recovered.

In some embodiments, the purification may be performed on a machine which functions based on the principle of a rotating syringe. The purification may be performed in an automated closed system such as a Sepax C-Pro Cell Processing or Sefia S2000 machine of Cytiva Europe. By closed system, it is to be understood a system wherein the suspension does not come into contact with the external environment during the density gradient purification. The closed system minimizes the risk of contamination and maintain cell viability

The method may further comprise cooling S400 the pancreatic cells, notably in order to optimize the viability of the purified pancreatic cells. Cooling S400 is performed during identifying S200 the optimum medium on the portion of the suspension and/or separating S300 the pancreatic cells. Cooling may also be performed in reducing S120 the digestate, washing S125 the reduced digestate and/or resuspending S130 the reduced digestate, in particular when the pancreatic cells are obtained through extraction of at least part of a pancreas. The pancreatic cells are preferably cooled to a temperature below 10 °C. Cooling S400 may comprise housing the cell processing machine in a coldroom or enclosed refrigerated unit, allowing processing at <10 °C.

In some embodiments, a cooling system can be used. The cooling system may be fitted to a system such as a Sepax C-Pro Cell Processing or Sefia S2000 machine of Cytiva Europe.

The cooling system may comprise a mechanically refrigerated heat exchanger and a dry medical air supply. The heat exchanger may comprise a nozzle configured to deliver cool air from the air supply to the cell processor or to an encasement built around the cell processor. The heat exchanger may further comprise a heater, positioned within the nozzle, configured to control the temperature of the air supply. Thus, medical air may be injected to the cell processor or through it's encasement with constant monitoring of the temperature.

The method may further comprise conditioning S500 the purified pancreatic cells for transplantation or usage. Conditioning S500 may comprise storing or cultivating S510 the purified pancreatic cells in bags, such as graft compatible blood bags, reducing in volume S520 the stored or cultivated purified pancreatic cells to reach a volume suitable for transplant (between 50 and 220 mL), washing S530 the reduced pancreatic cells suitable for transplant and S540 resuspending the reduced pancreatic cells in transplant bags. Conditioning S500 may be performed in an automated closed system, such as a machine from Cytiva Europe. In some examples, cooling S400 may be applied during conditioning S500.

In some examples, the stored or cultivated pancreatic cells may be directly transported to a distant graft center. Reducing in volume S520, washing S530 and resuspending 540 the pancreatic cells may take place at the distant graft center following transportation and prior to transplantation. The distant graft center may use a system such as a machine from Cytiva Europe.

Disclosed systems comprise a SEPAX™/SEFIA™platform.

Disclosed kits comprise systems comprising a SEPAX™/SEFIA™ platform.

Disclosed methods comprise:
(1) identification of human islet density to optimize purification for each donor and development of a specially adapted protocol for purification of islet cells using SEPAX™/SEFIA™ technology to replace the COBE 2991;
(2) development of a novel cooling system to allow processing at <10 °C for SEPAX™/SEFIA™ technology to protect human islets during processing and optimise the viability of purified islet cells;
(3) automated closed system volume reduction of pancreatic digestate to replace extensive manual centrifugation with SEFIA™ technology;
(4) automated closed system washing of pancreatic digestate and resuspension in UW solution in a closed system in preparation for purification to replace manual centrifugation with SEFIA™ technology;
(5) automated closed system washing of human islets in culture, reduction of volume, and resuspension in a blood bag of human islets in transplant medium with SEPAX™/SEFIA™ technology;

(6) SEPAX/SEFIA™ technology system is GMP compliant with cleanroom grade C&B;
(7) Using SEPAX™/SEFIA™ technology for islet cell processing require 1 staff only.

Disclosed methods further comprise production/processing of human pancreatic islet digestate for transplantation. For example, disclosed methods provide human islets suitable for transplantation.

Disclosed methods further comprise treatment of a disease or condition. For example, in embodiments, a diabetic patient can be treated, for example by administering a therapeutically effective amount of islet cells from cadaveric donors in the context of islet allografts, living donors in the context of islet autografts. Specific steps of the herein described processing further could be extended to pluripotent stem cell derived islets/insulin secreting cells or other cell therapies.

Disclosed systems and methods comprise replacement of the 1-2 COBE 2991 and the 1-2 large volume centrifuges currently employed for islet isolation by 1 for autografts and by 2 table top cGMP SEPAX™ 2 machines for islet allografts which take up less room, offering the unexpected advantage that no custom clean room will be necessary, and islet processing can take place in any standard clean room (9-12 m²). Implementation of volume reduction and washing, typically performed manually by centrifugation during processing of the digested pancreatic tissue, and post storage/culture requires 1 SEFIA™. Islet density gradient purification can be performed in the SEFIA™ in parallel with a second density gradient purification in the SEPAX™. Hence, full processing of pancreatic tissue (large volume >5 L volume reduction, washing, resuspension in UW) and including the density gradient purification step would thus require either 1 SEFIA™ and 1 SEPAX™ machines or alternatively 1 SEFIA™ and 2 SEPAX™ machines or 2 SEFIA™ machines, depending on the equipment preference and procedure streamlining of the technical staff and the center.

Disclosed systems and methods comprise reduction in islet cell production cost. For example, disclosed methods can reduce the number of required personnel from, for example, 3 to 2 people for the 6-7 hours typically required for the process.

Disclosed systems, kits, and methods provide significant advantages. for example, islet allotransplantation is regulated by the FDA in the USA as a drug, and thus compliance with CGMP manufacturing is required (https://www.fda.gov/media/147524/download Witkowski P J Clin Med. 2021 Jun. 29; 10(13):2878 PMID: 34209541, Piemonti L Transplant Int 2021, PMID: 34048106), and the SEPAX™ 2 and SEFIA™ falls within the definition of a cGMP compliant apparatus. This apparatus is unknown to people working in the islet field and most stem cell fields with the exception of hematopoetic cells (SEPAX™) and dendritic cells (SEFIA™).

In addition, islet manufacturing for clinical transplantation in cGMP cell therapy facilities often do not employ 100% designated personal but rather share personnel among the different stem cell applications, including hematopoetic stem cells. Thus, shared personnel between islets and other stem cell application may be advantageous in particular if the personnel is already routinely using the SEPAX™ 2 and similar technology of the SEFIA™.

Further, current SEPAX™ and SEFIA™ kits are not fully adapted to islet processing and new kits of the instant disclosure can be used on the SEPAX™ or SEFIA™ to optimally process islets from the digestate to the transplant bag. The various steps can be standardized in a SEPAX™ and SEFIA™ including the volume reduction of islets stored/cultured in bags, washing, and resuspension in GRAFT medium and in the graft compatible blood bag.

The disclosed systems, kits, and methods provide tremendous advantages in this technology. For example, no one has yet attempted to perform both the multiple steps involved in the procedure between the collagenase digested pancreas and transplantation with one apparatus, and no one has yet been able to propose a multistep apparatus that is cGMP with CE labeling. Current methods include volume reduction and washing during the procedure are done in an open phase with a centrifuge.

In contrast, the instant disclosure first provides an alternative to the COBE 2991 density purification which will come to an end, and in addition an alternative CGMP-compliant procedure for the collection of digestate with collagenase and washing including resuspension in the last wash with a second medium (the wash medium is replaced by UW solution) purification with density gradients, washing post purification to remove the density gradients and resuspension in a second medium for example but not limited to a culture medium for culture of cells at 37 °C or 22 °C or a cold preservation solution for cold storage as described (Noguchi H Transplantation 89; 47-54,2010). Resuspension of the cells can be done directly in the culture bag that is placed in the incubator or cold stored. Washing is routinely performed during culture in the first 12 h of culture, @24 h and every 24 h thereafter. This step is performed in an open system in the majority if not all labs in the world.

In contrast, by the herein described disclosure, islets for the graft can be processed for transplantation requiring volume reduction, washing, and resuspension in transplant medium; where cells/islets, can directly be recovered in a new closed transplant bag in the graft medium for immediate transportation for transplantation or shipping. If islets are to be transplanted in centers >6-8 hours distance from the islet processing site, islet bags can be shipped and these steps including volume reduction, washing, and resuspension in transplant medium and transplant bag can be performed directly in the cell therapy unit at the remote transplant site.

While the use of the SEPAX™ is known, the traditional technique used in the SEPAX™ with use of one standard density gradient as is performed for other applications in particular hematopoetic cells will not optimally work to purify heterogenous pancreatic tissue.

In contrast, the instant disclosure provides a customized approach for each pancreas after enzymatic digestion, where a test gradient is first performed to optimize both the recovery of a maximum number of islets but within a limited transplant compatible volume of cells (<10 mls of pellet) for both allografts and autografts.

Thus, the instant disclosure provides the first closed system for use in isolation and processing of islets and stem cell derived insulin-secreting cells.

### EXAMPLES

### Example 1—Isolation of Human Islet Cells Human islet cells are isolated using the following protocol:

1) identification of human islet density for each donor and development of a specially adapted protocol for purification of islet cells using SEPAX™ to replace the COBE 2991;
2) development of a novel cooling system to allow processing at <10 °C to protect human islets during processing and optimise the viability of purified islet cells;
3) automated closed system volume reduction of pancreatic digestate to replace extensive manual centrifugation with SEPAX™/SEFIA™ technology;
4) automated closed system washing of pancreatic digestate and resuspension in UW solution in a closed system in preparation for purification to replace manual centrifugation with SEPAX™/SEFIA™; and 5) automated closed system washing of human islets in culture, reduction of volume, and resuspension in a blood bag of human islets in transplant medium with SEPAX™/SEFIA™Technology.

### Example 2—Treatment of a Diabetic Patient

A therapeutically effective amount of the human islet cells isolated as in Example 1 are transplanted into a diabetic patient.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

### Items

Item 1: A cGMP-compliant method for isolation of human islet cells comprising:
   identification of human islet density for each donor and development of a specially adapted protocol for purification of islet cells using a SEPAX™ platform to replace the COBE 2991;
   development of a novel cooling system to allow processing at <10 C to protect human islets during processing and optimise the viability of purified islet cells;
   use of an automated closed system volume reduction of pancreatic digestate to replace extensive manual centrifugation with SEPAX™/SEFIA™ technology;
   use of an automated closed system washing of pancreatic digestate and resuspension in UW solution in a closed system in preparation for purification to replace manual centrifugation with SEPAX™/SEFIA™ technology; and
   use of automated closed system washing of human islets in culture, reduction of volume, and resuspension in a blood bag of human islets in transplant medium.
Item 2: A method of treating a patient in need thereof, comprising administering a therapeutically effective amount of islet cells produced by the method of claim 1.
Item 3: A cGMP compliant system for isolation of islet cells, said system comprising a SEPAX™/SEFIA™ technology platform.
Item 4: A kit for isolation of islet cells, said kit comprising a SEPAX™/SEFIA™ technology platform and instructions for use.

1. A method of isolating pancreatic cells, comprising:
obtaining a suspension comprising biological material including pancreatic cells;
identifying an optimum medium for purifying the pancreatic cells;
separating the pancreatic cells from other biological material using the identified optimum medium, thereby isolating the pancreatic cells;
wherein identifying the optimum medium for purifying the pancreatic cells comprises performing a density gradient centrifugation on a portion of the suspension using a plurality of media, and identifying the optimum medium as the medium comprising pancreatic cells or the medium below an interface comprising pancreatic cells,
and wherein separating the pancreatic cells comprises performing a purification using the optimum medium, thereby purifying the pancreatic cells.

2. The method of claim 1, wherein the pancreatic cells are islet cells.

3. The method of claim 1, wherein the biological material is obtained through extraction from at least part of a pancreas or through differentiation of stem cells.

4. The method of claim 3, wherein the biological material is obtained through extraction, and wherein obtaining a suspension comprises:
digesting at least part of a pancreas to obtain a digestate; and
reducing the digestate in volume, washing the reduced digestate in a wash medium, and resuspending the reduced digestate in the wash medium or another medium.

5. The method of claim 4, wherein reducing the digestate in volume and washing the reduced digestate is performed in an automated closed system in which the digestate does not come into contact with the external environment during reduction of the digestate.

6. The method of claim 4, wherein obtaining a suspension further comprises dividing resuspended digestate in one or a plurality of bags for subsequent use, storage or preincubation.

7. The method of claim 1, wherein density gradient centrifugation on a portion of the suspension is performed using a discontinuous gradient.

8. The method of claim 7, wherein the discontinuous gradient of the portion of the suspension comprises a plurality of fractions of different densities.

9. The method of claim 8, wherein seven fractions are used.

10. The method of claim 1, wherein density gradient centrifugation on a portion of the suspension uses a dye.

11. The method of claim 1, wherein the density gradient centrifugation on a portion of the suspension is performed using top loading or bottom loading of the portion of the suspension in the plurality of media.

12. The method of, wherein identifying the optimum medium for purifying the pancreatic cells comprises identifying a second medium distinct from the optimum medium, the second medium comprising pancreatic cells or being a second medium below a second interface comprising pancreatic cells.

13. The method of claim 1, wherein the purification is performed in a machine that functions using a rotating syringe principle.

14. The method of claim 1, further comprising cooling the pancreatic cells.

15. The method of claim 1, further comprising conditioning purified pancreatic cells for transplantation or usage, on site or at a distant center.

16. A system for implementing the method of claim 1, the system comprising at least one automated closed system.

17. A method of treating a patient in need thereof, comprising administering a therapeutically effective amount of pancreatic cells isolated by the method of claim 1.

18. The method of claim 17, wherein the subject is diabetic.

19. The method of claim 10, wherein the dye is dithizone.
